(19) United States
(12) Patent Application Publication
(10) Pub. No.: US 2016/0045410 A1
(43) Pub. Date: Feb. 18, 2016

(54) Composition For The Treatment Of Hyperhidrosis

(71) Applicant: Michael Brier, Sunny Isles Beach, FL (US)

(72) Inventor: Michael Brier, Sunny Isles Beach, FL (US)

(21) Appl. No.: 14/461,602

(22) Filed: Aug. 18, 2014

(51) Int. Cl. A61K 8/26; A61K 8/86; A61K 8/02; A61K 8/34; A61Q 15/00; A61Q 17/00; A61K 8/97; A61K 8/72

(52) U.S. Cl. CPC A61K 8/26; A61K 8/97; A61K 8/86; A61K 8/72; A61K 8/345; A61K 8/347; A61Q 15/00; A61Q 17/005; A61K 8/0208; A61K 2800/87; A61K 2800/5922

(57) ABSTRACT

An inventive composition for the treatment of hyperhidrosis comprises an active ingredient, namely aluminum chloride in the range of 14.25% to 15.75% by weight and water in the range of about 75% to 80% by weight. The solution of the inventive composition is applied to a body via a pre-soaked towelette by dabbing the towelette over the parts of the body susceptible to excessive sweating. The composition includes a skin protein that binds with the active ingredient to effectively block the pores and sweat glands to control excessive perspiration. The solution of the composition is applied in the evening before bedtime, and immediately after showering in hot water, so that the pores are most receptive to the solution and the composition effectively blocks the pores.

### BACKGROUND OF THE INVENTION

1. Field of the Invention

The invention relates generally to the treatment for hyperhidrosis. In particular, the invention relates to a personal wipe impregnated with a quick drying antiperspirant formulation to control cosmetic and excessive sweating, wherein the formulation includes aluminum chloride in the range of 15%, a humectant, skin conditioner agents and a skin protein. The waterproof formulation is applied to the body via a wipe, in the evening and continues to be effective for up to eight days.

2. Description of Related Art

Perspiration serves as the body's coolant mechanism. Perspiration protects the body from overheating and is essential for survival. Hyperhidrosis is a condition characterized by excessive sweating or perspiration, beyond that which is required for normal regulation of body temperature. In some instances, an individual with hyperhidrosis may sweat four or five times more than is necessary or normal. Individuals afflicted with excessive sweating often feel psychologically, emotionally or socially burdened. Hyperhidrosis can either be generalized or localized to specific parts of the body. Hands, feet, armpits, and the groin area are among the most active regions of perspiration due to the relatively high concentration of sweat glands.

In severe cases, physicians may suggest surgery to remove the sweat glands or o disconnect the nerves responsible for the overproduction of sweat. Many complications may result as a consequence of hyperhidrosis, including skin infections, ranging from ringworm warts. In addition, other skin conditions, such as rashes or eczema have been found to occur more often in people with hyperhidrosis.

Hyperhidrosis may also be treated with topical formulations. U.S. Pat. No. 8,618,160 (to Johnston et al.) discloses individually packaged topical formulations comprising about 0.25 to about 6% glycopyrrolate for the treatment of hyperhidrosis, wherein said wipe is contained within a pouch resistant to leakage.

U.S. Pat. No. 5,403,588 (to Santa Ana, Jr.) teaches a body deodorant composition consisting essentially of a combination of 70% isopropyl alcohol, acetone and aluminum chloride in individual amounts which provide cleansing and deodorizing effects. The body deodorizing composition is incorporated into an absorbent cotton pad as a solution and packaged in individual pouches for use.

Thus, there is a need for an effective and long lasting antiperspirant composition that may be used all over a human body to control hyperhidrosis wherein the formulation begins to work immediately when applied and also avoids the skin irritation and chafing chafing that results with antiperspirant formulations.

### SUMMARY OF THE INVENTION

The present invention is directed to an antiperspirant composition for the treatment of hyperhidrosis that comprises 14.25% to 15.75% by weight of an active deodorant. The composition further includes a skin protein that binds with the active deodorant to effectively block the pores and sweat glands to control excessive perspiration. The composition further comprises 75% to 80% water by weight. The composition of the present invention includes a humectant to reduce the loss of water and a combination of anti-inflammatory and skin conditioning agents to reduce skin irritation from the active deodorant.

In addition, the composition of the present invention includes 1% to 5% by weight of an emulsifier. The composition further comprises a combination of antimicrobial, antiseptic and antioxidant agents.

In a preferred embodiment, a substrate, such as a non woven soft towelette is pre-soaked with a predetermined amount of the antiperspirant composition of the present invention. The composition is applied to the body by dabbing, not rubbing or wiping, the pre-soaked towelette or pad over the body. The composition may be applied anywhere over the body, from head to toe, including the facial area, with the exception of the area around the eyes.

The composition of the present invention is a quick drying formulation and begins to work on controlling perspiration immediately when applied.

An object of the present invention is to control cosmetic and excessive sweating and associated complications, including body odor, sweat, rashes, athletes foot, warts and chafing.

Another object of the present invention is to provide a quick drying formulation that will prevent odor caused by moisture emitting bacteria that accompanies excessive sweating.

Yet another object of the present invention is to provide a treatment for hyperhidrosis, in a convenient foil packet, containing a single application towelette that has been pre-soaked with the composition of the present invention.

Another object of the present invention is to provide a formulation that forms a block in the pores to obstruct the sweat glands in order to control excessive sweating for a period of up to seven days.

An advantage of the composition of the present invention is that it begins to work immediately when applied.

Other advantages of the composition of the present invention are that it is does not form a visible film when applied, is waterproof, does not need to be reapplied after showering, and will remain effective for up to seven days.

Yet another advantage of the composition of the present invention is that it includes vitamin E and aloes to reduce skin irritation.

Another advantage of the present invention is that it may be applied all over the body, to control sweating, including the scalp, hands, feet, groin, buttocks, back and torso.

It is noted that references made herein to the present invention or aspects of the invention thereof should be understood to mean certain embodiments of the present invention and should not necessarily be construed as limiting all embodiments to a particular description. The present invention is set forth in various levels of detail in the Summary of the Invention as well as in the attached drawings and the Detailed Description of the Invention and no limitation as to the scope of the present invention is intended by either the inclusion or non-inclusion of elements, components, etc. in this Summary of the Invention. Additional aspects of the present invention will become more readily apparent upon further review of the Specification.

### DETAILED DESCRIPTION OF THE INVENTION

The present inventive composition and method of use, provide an improvement to sufferers of hyperhidrosis over the prior art. The composition of the present invention combines a carrier and an active deodorant with an emulsifier, a skin protein, an anti-inflammatory agent, a humectant, an anti-oxidant, an anti-microbial agent and antiseptic, with a combination of skin conditioning agents, to produce a unique composition for the treatment of hyperhidrosis, that is waterproof, non irritating and effective in controlling excessive sweating for a period of up to seven days, with a single application.

In a preferred embodiment, a non-woven soft towelette or pad is pre-soaked with a solution of the inventive composition for a single application. The pre-soaked towelette or pad is sealed in an air tight aluminum foil pouch that is resistant to leakage, to preserve its effectiveness.

In a preferred method of use, the treatment is effected by applying the composition of the present invention to one or more areas of the body that is susceptible to excessive sweating. The composition may be applied anywhere on the body, with the exception of the eye area. Areas of the body that may be afflicted with hyperhidrosis include the head, scalp, torso, back, hands, feet, buttocks and groin area.

Preferably, the composition is applied directly to the body after showering, in the evening, and just before bedtime. The composition is applied by dabbing the pre-soaked towelette over one or more areas of the body to be treated. It is noted that the composition should not be rubbed into the skin, nor should the towelette be wiped over the area of the body to be treated. Rather, the preferred method of application is to dab the composition, by lightly pressing the pre-soaked towelette to the skin. The technique of lightly pressing the composition to the skin via the pre-soaked towelette is preferred as it minimizes irritation.

Preferably, the composition is applied in the evening before bedtime. During the night, while the user is asleep, the composition will form a block over the pores of the sweat glands that will last up to seven days and will not wash off and remain waterproof, through showering.

The composition of the present invention is quick drying and will not leave any film on the skin. The quick drying composition of the present invention is formed from a combination of an active deodorant, in particular aluminum chloride and a skin protein. The combination produces a composition that effectively plugs the pores of the sweat glands to control excessive sweating caused by hyperhidrosis. The inventive composition is not irritating and will begin to work immediately to block sweat wherever it is applied.

The antiseptic and antimicrobial agents of the inventive composition combat odor caused by moisture emitting bacteria.

The inventive composition comprises a solution of aluminum chloride in water, wherein aluminum chloride is the active ingredient for the deodorant, and wherein the aluminum chloride is present in the range of 14.25% to 15.75% by weight. The composition of the present invention comprises a carrier, preferably water in the range of about 75% to 80% by weight. A humectant, preferably, polyethylene glycol or PEG-180 is present in a range of 0.10% to 0.50% by weight. PEG-180 is beneficial as it acts as a humectant and solvent in the solution.

The composition further includes a skin protein that binds with the active ingredient, that is, aluminum chloride, to effectively block the pores and sweat glands to control excessive perspiration. Allantoin is present in a preferred range of 0.10% to 0.50% by weight.

The composition of the present solution incorporates a surfactant, to act as an emulsifier. In a preferred embodiment, the composition of the present invention includes a polysorbate, namely polysorbate 20 in a range of about 1% to 5% by weight. An anti-inflammatory agent is desirable to soothe the skin in view of the comparatively high concentration of aluminum chloride. Preferably, the solution of the present invention comprises aloe barbadensis leaf juice in a range of 1% to 5% by weight.

The composition of the present invention further comprises a skin conditioning agent. Preferably the skin conditioning agent includes antibacterial properties for preventing infection. In a preferred embodiment, caprylyl glycol is used in a range of 0.10% to 1.00% by weight. Caprylyl glycol contains two primary hydroxyl groups with both bacteriostatic , that is, capable of inhibiting the growth of bacteria and bactericidal, that is, preventing infection by inhibiting the growth of bacteria properties.

An antiseptic agent, such as eugenol, is present in the preferred range of 0.10% to 0.50% by weight. It has been found that it is especially effective to include a compound for controlling microbial growth that is particularly effective in aqueous solutions. In particular, methylisothiazolinone is utilized in the composition in an amount less than 0.10% by weight for controlling microbial growth in water containing solution.

A stable antioxidant in a preferred range of 0.10% to 0.50% by weight is present in the composition of the present invention. It has been determined that an ester of acetic acid and tocopherol is preferred in the present composition because it exhibits more stable and less acidic qualities than pure tocopherol. In particular, tocopheryl acetate is used in a preferred embodiment, because in addition to being an antioxidant it has the added benefit of acting as a skin conditioning agent.

Glycerin, in a preferred range of 0.50% to 1.00% by weight is present in the composition of the present invention as a lubricating agent.

A high concentration of aluminum chloride in the composition of the present invention may be irritating to human skin. Therefore, it has been found that the composition of the present invention benefits from the presence of a compound that exhibits anti-inflammatory properties. In addition to exhibiting beneficial anti-inflammatory properties, avena sativa (oat) kernel extract acts as a skin conditioning agent and anti-oxidant. In a preferred embodiment, avena sativa (oat) kernel extract is utilized in the composition in an amount less than 0.10% by weight.

A combination of skin conditioning agents, namely althaea officinalis root extract, cucumis sativus, chamomilla recutita and aloe barbadensis leaf extract, are each present in the composition in an amount less than 0.10% by weight.

Thus, the synergistic effect of this combination of ingredients, results in a uniquely effective and soothing treatment for hyperhidrosis.

Thus, while there has been shown and described, fundamental novel features of the disclosure as applied to various specific embodiments thereof, it will be understood that various omissions and substitutions and changes in the form and details of the apparatus illustrated, and in their operation, may be made by those skilled in the art without departing from the spirit of the disclosure. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A deodorant composition for the treatment of hyperhidrosis comprising:
(a) an active deodorant in a range from about 14.25% to about 15.75% by weight;
(b) a carrier liquid in a range from about 75.00% to about 80.00% by weight;
(c) a surfactant in a range from about 1.00% to about 5.00% by weight; and
(d) a first anti-inflammatory agent in a range from about 1.00% to about 5.00% by weight.

2. The composition of claim 1, further comprising a humectant, wherein the humectant is present in a range from about 0.10% to about 0.50% by weight.

3. The composition of claim 1, further comprising a skin protein to bind with the active deodorant, wherein the skin protein is present in a range from about 0.10% to about 0.50% by weight.

4. The composition of claim 1, further comprising a skin conditioning agent, wherein the skin conditioning agent further includes antibacterial properties for preventing infection, wherein the skin conditioning agent is present in a range from about 0.10% to about 1.00% by weight.

5. The composition of claim 1, further comprising an antiseptic agent, wherein the antiseptic agent is present in a range from about 0.10% to about 0.50% by weight.

6. The composition of claim 1, further comprising a stable antioxidant, wherein the antioxidant is present in a range from about 0.10% to about 0.50% by weight.

7. The composition of claim 1, further comprising a lubricating agent, wherein the lubricating agent is present in a range from about 0.50% to about 1.00% by weight.

8. The composition of claim 1, further comprising a second anti-inflammatory agent, wherein the second anti-inflammatory agent further includes skin conditioning and anti-oxidant, wherein the second anti-inflammatory agent is present in an amount less than about 0.10% by weight.

9. The composition of claim 1, further comprising an anti-microbial agent, present in an amount less than about 0.10% by weight, wherein the anti-microbial agent controls microbial growth in water containing solutions.

10. The composition of claim 1, further comprising a combination of skin conditioning agents, wherein the combination of skin conditioning agents is althaea officinalis root extract in an amount less than about 0.10% by weight, cucumis sativus in an amount less than about 0.10% by weight, chamomilla recutita in an amount less than about 0.10% by weight, and aloe barbadensis leaf extract in an amount less than about 0.10% by weight.

11. A substrate for the treatment of hyperhidrosis, wherein the substrate is impregnated with a deodorant composition comprising:
(a) aluminum chloride a range from about 14.25% to about 15.75% by weight;
(b) water in a range from about 75.00% to about 80.00% by weight;
(c) polysorbate 20 in a range from about 1.00% to about 5.00% by weight;
(d) aloe barbadensis leaf juice in a range from about 1.00% to about 5.00% by weight;
(e) polyethylene glycol 180 in a range from about 0.10% to about 0.50% by weight;
(f) allantoin in a range from about 0.10% to about 0.50% by weight;
(g) caprylyl glycol in a range from about 0.10% to about 1.00% by weight;
(h) eugenol in a range from about 0.10% to about 0.50% by weight;
(i) tocopheryl acetate in a range from about 0.10% to about 0.50% by weight;
(k) glycerin in a range from about 0.50% to about 1.00% by weight;
(l) avena sativa (oat) kernel extract in an amount less than about 0.10% by weight;
(m) methylisothiazolinone in an amount less than about 0.10% by weight;
(n) a Ithaca officinalis root extract in an amount less than about 0.10% by weight;
(o) cucumis sativus in an amount less than about 0.10% by weight;
(p) chamomilla recutita in an amount less than about 0.10% by weight; and
(q) aloe barbadensis leaf extract in an amount less than about 0.10% by weight.

12. A method of treating hyperhidrosis wherein the method comprises the following steps:
(a) preparing one or more areas of a body of the user by removing dirt, residue and pore clogging debris from the body with a cleansing agent and warm water, wherein the warm water aids in the cleansing and further serves to open skin pores on the body;
(b) thoroughly rinsing the body of the cleansing agent;
(c) completely drying the body with an absorbent towel;
(d) providing a user with a towelette, wherein the towelette is impregnated with a composition for the treatment of hyperhidrosis;
(e) dabbing the towelette on one or more areas of the body to cover and penetrate the skin pores; and
(f) allowing the body to remain in a restful state in order to allow the composition to effectively block the pores and sweat glands to control excessive perspiration.

13. The method of claim 12, wherein the treatment is effective at controlling excessive sweating for a period of seven days.

14. The method of claim 12, wherein the treatment does not require reapplication and continues to be effective after bathing or showering for a period of seven days.

15. The method of claim 12, wherein the towelette is impregnated with a compositing comprising:
(a) an active deodorant in a range from about 14.25% to about 15.75% by weight;
(b) a carrier liquid in a range from about 75.00% to about 80.00% by weight;
(c) a surfactant in a range from about 1.00% to about 5.00% by weight; and
(d) an anti-inflammatory agent in a range from about 1.00% to about 5.00% by weight.

16. The method of claim 15, wherein the active deodorant is aluminum chloride, the carrier liquid is water, the surfactant is polysorbate 20 and the anti-inflammatory agent is aloe barbadensis leaf juice.
